(19) United States
(12) Patent Application Publication
(10) Pub. No.: US 2003/0198597 A1
(43) Pub. Date: Oct. 23, 2003

(54) Novel macrocyclic activatible magnetic resonance imaging contrast agents

(76) Inventors: Thomas J. Meade, Altadena, CA (US); Douglas A. Bakan, San Diego, CA (US)

Correspondence Address: Robin M. Silva, DORSEY & WHITNEY LLP, Suite 3400, Four Embarcadero Center, San Francisco, CA, 94111-4187, US

(21) Appl. No.: 10/131,616

(22) Filed: Apr. 22, 2002

(51) Int. Cl. A61K 49/00; C07F 5/00; C07H 17/02; C07K 14/47

(52) U.S. Cl. 424/9.34; 424/9.35; 530/400; 534/15; 536/17.4

(57) ABSTRACT

The invention relates to novel magnetic resonance imaging contrast agents and methods of detecting physiological signals or substances.

### FIELD OF THE INVENTION

The invention relates to novel magnetic resonance imaging contrast agents and methods of detecting physiological signals or substances.

### BACKGROUND OF THE INVENTION

Magnetic resonance imaging (MRI) is a diagnostic and research procedure that uses high magnetic fields and radio-frequency signals to produce images. The most abundant molecular species in biological tissues is water. It is the quantum mechanical “spin” of the water proton nuclei that ultimately gives rise to the signal in all imaging experiments. In MRI the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. MRI is able to generate structural information in three dimensions in relatively short time spans.

The Image.

MR images are typically displayed on a gray scale with black the lowest and white the highest measured intensity (I). This measured intensity I=C*M, where C is the concentration of spins (in this case, water concentration) and M is a measure of the magnetization present at time of the measurement. Although variations in water concentration (C) can give rise to contrast in MR images, it is the strong dependence of the rate of change of M on local environment that is the source of image intensity variation in MRI. Two characteristic relaxation times, T₁ & T₂, govern the rate at which the magnetization can be accurately measured. T₁ is the exponential time constant for the spins to decay back to equilibrium after being perturbed by the RF pulse. In order to increase the signal-to-noise ratio (SNR) a typical MR imaging scan (RF & gradient pulse sequence and data acquisition) is repeated at a constant rate for a predetermined number of times and the data averaged. The signal amplitude recorded for any given scan is proportional to the number of spins that have decayed back to equilibrium since the previous scan. Thus, regions with rapidly decaying spins (i.e. short T₁ values) will recover all of their signal amplitude between successive scans.

The measured intensities in the final image will accurately reflect the spin density (i.e. water content). Regions with long T₁ values compared to the time between scans will progressively lose signal until a steady state condition is reached and will appear as darker regions in the final image. Changes in T₂ (spin-spin relaxation time) result in changes in the signal linewidth (shorter T₂ values) yielding larger linewidths. In extreme situations the linewidth can be so large that the signal is indistinguishable from background noise. In clinical imaging, water relaxation characteristics vary from tissue to tissue, providing the contrast which allows the discrimination of tissue types. Moreover, the MRI experiment can be setup so that regions of the sample with short T₁ values and/or long T₂ values are preferentially enhanced so called T₁-weighted and T₂-weighted imaging protocol.

MRI Contrast Agents.

There is a rapidly growing body of literature demonstrating the clinical effectiveness of paramagnetic contrast agents (currently 8 are in clinical trials or in use). The capacity to differentiate regions/tissues that may be magnetically similar but histologically distinct is a major impetus for the preparation of these agents [1, 2]. In the design of MRI agents, strict attention must be given to a variety of properties that will ultimately effect the physiological outcome apart from the ability to provide contrast enhancement [3]. Two fundamental properties that must be considered are biocompatability and proton relaxation enhancement. Biocompatability is influenced by several factors including toxicity, stability (thermodynamic and kinetic), pharmacokinetics and biodistribution. Proton relaxation enhancement (or relaxivity) is chiefly governed by the choice of metal and rotational correlation times.

The first feature to be considered during the design stage is the selection of the metal atom, which will dominate the measured relaxivity of the complex. Paramagnetic metal ions, as a result of their unpaired electrons, act as potent relaxation enhancement agents. They decrease the T₁ and T₂ relaxation times of nearby (r⁶ dependence) spins. Some paramagnetic ions decrease the T₁ without causing substantial linebroadening (e.g. gadolinium (III), (Gd³⁺)), while others induce drastic linebroadening (e.g. superparamagnetic iron oxide). The mechanism of T₁ relaxation is generally a through space dipole-dipole interaction between the unpaired electrons of the paramagnet (the metal atom with an unpaired electron) and bulk water molecules (water molecules that are not “bound” to the metal atom) that are in fast exchange with water molecules in the metal's inner coordination sphere (are bound to the metal atom).

For example, regions associated with a Gd³⁺ ion (near-by water molecules) appear bright in an MR image where the normal aqueous solution appears as dark background if the time between successive scans in the experiment is short (i.e. T₁ weighted image). Localized T₂ shortening caused by superparamagnetic particles is believed to be due to the local magnetic field inhomogeneities associated with the large magnetic moments of these particles. Regions associated with a superparamagnetic iron oxide particle appear dark in an MR image where the normal aqueous solution appears as high intensity background if the echo time (TE) in the spin-echo pulse sequence experiment is long (i.e. T₂-weighted image). The lanthanide atom Gd³⁺ is by the far the most frequently chosen metal atom for MRI contrast agents because it has a very high magnetic moment (u²=63 BM²), and a symmetric electronic ground state, (S⁸). Transition metals such as high spin Mn(II) and Fe(III) are also candidates due to their high magnetic moments.

Once the appropriate metal has been selected, a suitable ligand or chelate must be found to render the complex nontoxic. The term chelator is derived from the Greek word chele which means a “crabs claw”, an appropriate description for a material that uses its many “arms” to grab and hold on to a metal atom (see DTPA below). Several factors influence the stability of chelate complexes include enthalpy and entropy effects (e.g. number, charge and basicity of coordinating groups, ligand field and conformational effects). Various molecular design features of the ligand can be directly correlated with physiological results. For example, the presence of a single methyl group on a given ligand structure can have a pronounced effect on clearance rate. While the addition of a bromine group can force a given complex from a purely extracellular role to an effective agent that collects in hepatocytes.

Diethylenetriaminepentaacetic (DTPA) chelates and thus acts to detoxify lanthanide ions. The stability constant (K) for Gd(DTPA)²⁻ is very high (logK=22.4) and is more commonly known as the formation constant (the higher the logK, the more stable the complex). This thermodynamic parameter indicates the fraction of Gd³⁺ ions that are in the unbound state will be quite small and should not be confused with the rate (kinetic stability) at which the loss of metal occurs (k_{f}/k_{d}). The water soluble Gd(DTPA)²⁻ chelate is stable, nontoxic, and one of the most widely used contrast enhancement agents in experimental and clinical imaging research. It was approved for clinical use in adult patients in June of 1988. It is an extracellular agent that accumulates in tissue by perfusion dominated processes.

To date, a number of chelators have been used, including diethylenetriaminepentaacetic (DTPA), 1,4,7,10-tetraazacyclododecane′-N,N′N″,N′″-tetracetic acid (DOTA), and derivatives thereof. See U.S. Pat. Nos. 5,155,215, 5,087,440, 5,219,553, 5,188,816, 4,885,363, 5,358,704, 5,262,532, and Meyer et al., Invest. Radiol. 25: S53 (1990).

Image enhancement improvements using Gd(DTPA) are well documented in a number of applications (Runge et al., Magn, Reson. Imag. 3:85 (1991); Russell et al., AJR 152:813 (1989); Meyer et al., Invest. Radiol. 25:S53 (1990)) including visualizing blood-brain barrier disruptions caused by space occupying lesions and detection of abnormal vascularity. It has recently been applied to the functional mapping of the human visual cortex by defining regional cerebral hemodynamics (Belliveau et al., (1991) 254:719).

Another chelator used in Gd contrast agents is the macrocyclic ligand 1,4,7,10-tetraazacyclododecane-N,N′,N″N′″-tetracetic acid (DOTA). The Gd-DOTA complex has been thoroughly studied in laboratory tests involving animals and humans. The complex is conformationally rigid, has an extremely high formation constant (logK=28.5), and at physiological pH possess very slow dissociation kinetics. Recently, the GdDOTA complex was approved as an MRI contrast agent for use in adults and infants in France and has been administered to over 4500 patients.

Previous work has resulted in MRI contrast agents that report on physiologic or metabolic processes within a biological or other type of sample. As described in U.S. Pat. No. 5,707,605, PCT US96/08549, and U.S. Ser. No. 09/134,072, MRI contrast agents have been constructed that allow an increase in contrast as a result of the interaction of a blocking moiety present on the agent with a target substance. That is, in the presence of the target substance, the exchange of water in one or more inner sphere coordination sites of the contrast agent is increased, leading to a brighter signal; in the absence of the target substance, the exchange of water is hindered and the image remains dark. Thus, the previous work enables imaging of physiological events rather than just structure.

Accordingly, it is an object of the invention to provide further examples of activatable or functional MRI contrast agents for the detection of physiological substances.

### SUMMARY OF THE INVENTION

In accordance with the objects outlined above, the present invention provides chelates having the formula:

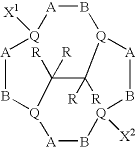

wherein

each Q is independently selected from the group consisting of nitrogen, oxygen or sulfur;

A—B is a structure selected from the group consisting of —CR₂—CR₂—, —CR═CR—, —CR₂—CR₂—CR₂—, —CR═CR—CR₂— and —CR₂—CR═CR—;

X1 and X2 are independently selected from the group consisting of CR₂COO⁻, CR₂COOH, CR₂(BM), CR(CR₂COO⁻)₂, CR(CR₂COO⁻)(BM), CR(CR₂COOH)₂ and CR(CR₂COOH)(BM), wherein BM is a blocking moiety; and

each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, targeting moiety, blocking moiety, or, together with an adjacent R group forms an alkyl or aryl group;

wherein either:

a) X¹ or X² comprises a BM; or

b) at least one R comprises a BM.

In an additional aspect, the present invention provides chelates having the formula:

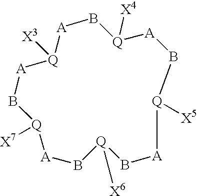

wherein

each Q is independently selected from the group consisting of nitrogen, oxygen or sulfur;

A—B is a structure selected from the group consisting of —CR₂—CR₂—, —CR═CR—, —CR₂—CR₂—CR₂—, —CR═CR—CR₂— and —CR₂—CR═CR—;

X³, X⁴, X⁵, X⁶ and X⁷ are independently selected from the group consisting of —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3, wherein BM is a blocking moiety;

each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, blocking moiety, or, together with an adjacent R group forms an alkyl or aryl group;

wherein optionally two of X³—X⁷ are joined to form —CR₂—CR₂—, —CR═CR—, —CR₂—CR₂—CR₂— or —CR═CR—CR₂—; and

wherein either:

a) X³, X⁴, X⁵, X⁶ or X⁷ comprises a BM; or

b) at least one R comprises a BM.

In a further aspect, the invention provides chelates having the formula:

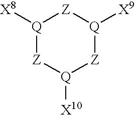

wherein

each Q is independently selected from the group consisting of nitrogen, sulfur or oxygen;

each Z is —(CR₂)n-wherein n is at least 1 and R is a substitution group;

at least two of X⁸—X¹⁰ are selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3;

wherein at least one R or X comprises a blocking moiety.

In an additional aspect, the invention provides chelates having the formula:

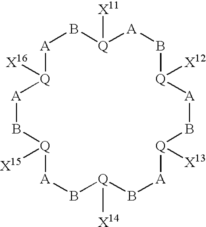

wherein A and B are selected from the group consisting of CR₂—CR₂, CR═CR, CR₂—CR₂—CR₂, CR═CR—CR₂, and CR₂—CR═CR;

each Q is independently selected from the group consisting of nitrogen, sulfur or oxygen;

each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, blocking moiety, or, together with an adjacent R group forms an alkyl or aryl group, or together with an non adjacent R group forms an alkyl or aryl group;

wherein X¹¹—X¹⁶ are selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻) (BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3;

wherein optionally two of X¹—X⁷ are joined to form a C1-5 alkyl group;

wherein either:

a) X¹¹, X¹², X¹³, X¹⁴, X¹⁵ or X¹⁶ comprises a BM; or

b) at least one R comprises a BM.

In a further aspect, the invention provides chelates having the formula:

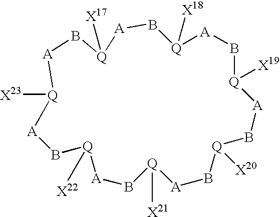

wherein

A and B are selected from the group consisting of CR₂—CR₂, CR═CR, CR₂—CR₂—CR₂, CR═CR—CR₂, and CR₂—CR═CR;

each Q is independently selected from the group consisting of nitrogen, sulfur or oxygen;

each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, blocking moiety, or, together with an adjacent R group forms an alkyl or aryl group, or together with an non adjacent R group forms an alkyl or aryl group;

wherein each of X²³ are independently selected from the group consisting of hydrogen, R, blocking moiety,—(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3;

wherein optionally two of X¹⁷—X²³ are joined to form a C1-10 alkyl group;

wherein either:

a) X¹⁷, X¹⁸, X¹⁹, X²⁰, X²¹, X²² or X²³ comprises a BM; or

b) at least one R comprises a BM.

In a further aspect, the invention provides any of the chelates of the invention further comprising a paramagnetic metal ion, and methods of imaging using the complexes.

### BRIEF DESCRIPTION OF THE DRAWINGS

FIGS. 1A-1Q depict a variety of preferred embodiments utilizing macrocycles with four coordination atoms in the macrocycle. FIG. 1A depicts Structure 1 of the invention. FIG. 1B depicts the chelator of FIG. 1A with nitrogen atoms as the coordination atoms. FIGS. 1C-1I depict a variety of preferred structures, using nitrogen as the coordination atoms, a simple alkyl as the —A—B— group, with X “basket” linkages, and without any additional R groups. FIGS. 1J-1Q depict a variety of structures with a variety —A—B— linkages and no additional R groups, of which some include the X “basket” linkages. As will be appreciated by those in the art, the X groups on the X-linked structures can include “branched arms”. None of depicted chelates show the metal ions.

FIGS. 2A-2E depict a variety of suitable —A—B— groups, any one of which can be included in the macrocycles of the invention.

FIGS. 3A and 3B depict two preferred embodiments utilizing macrocycles with five coordination atoms in the macrocycle. None of depicted chelates show the metal ions.

FIG. 4 depicts a preferred embodiment utilizing macrocycles with three coordination atoms in the macrocycle. The depicted chelate does not show the metal ion.

FIG. 5 depicts a chelate with six coordination atoms. The depicted chelate does not show the metal ion, and as will be appreciated by those in the art, more than one metal ion may be coordinated in these structures.

FIG. 6 depicts chelates with seven coordination atoms. None of depicted chelates show the metal ions, and as will be appreciated by those in the art, more than one metal ion may be coordinated in these structures.

FIGS. 7A-7J depict a number of suitable X “arms” for the chelates and compositions of the invention. As will be appreciated by those in the art, n is an integer from 1 to 10, with 1 to 2 being preferred. In some preferred embodiments, one or more of the —COOH groups can be replaced by either a blocking moiety (including a coordination site barrier), a targeting moiety, or, in some cases, any R group.

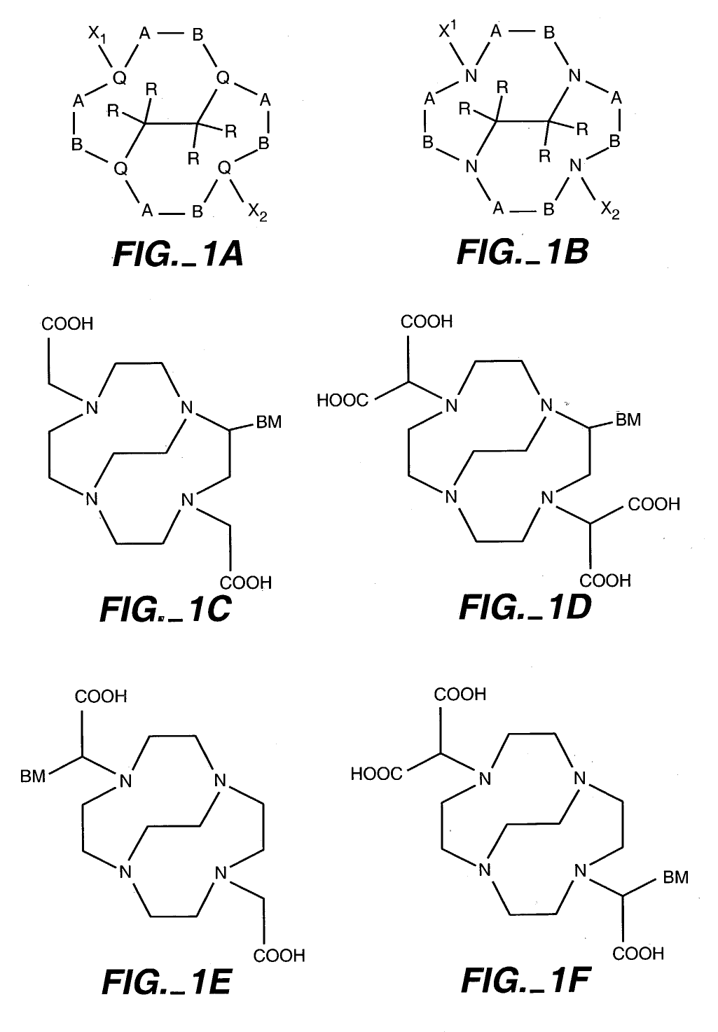

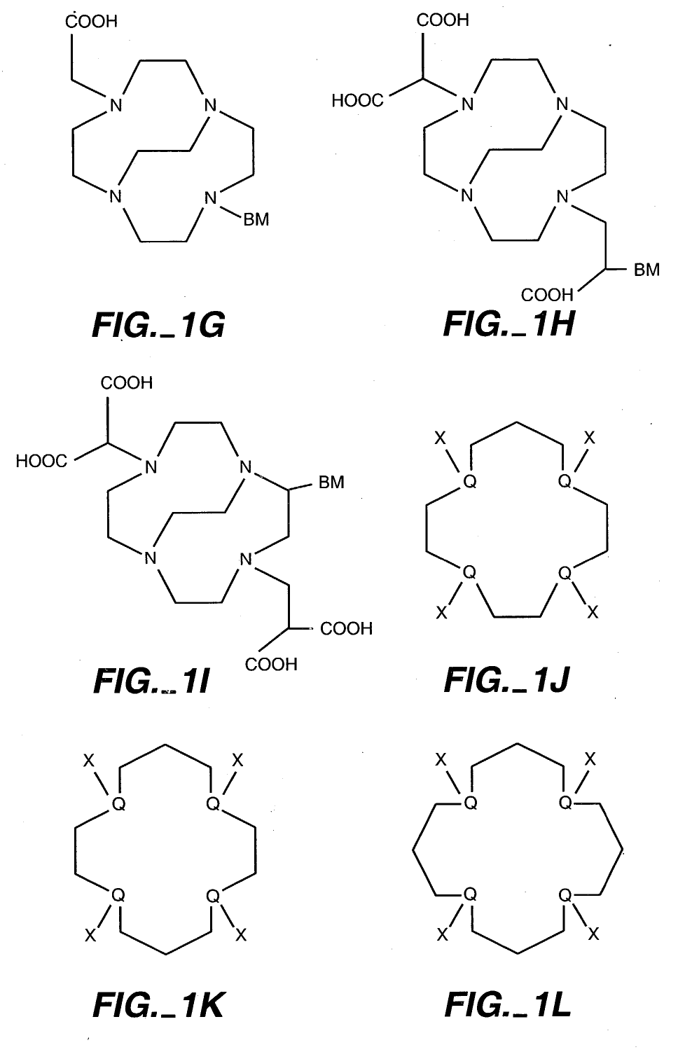

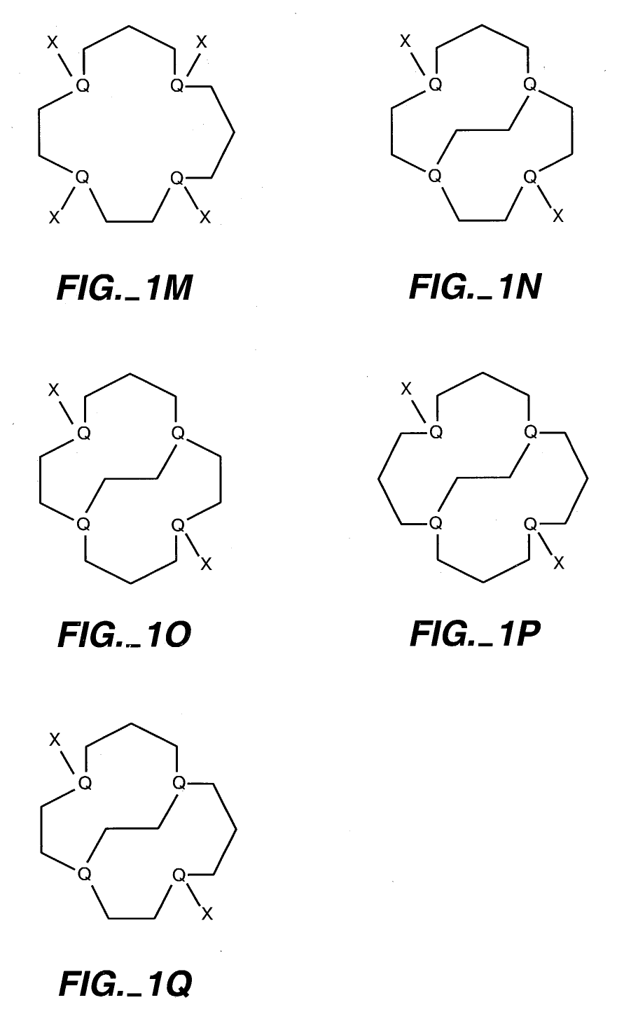

### DETAILED DESCRIPTION OF THE INVENTION

The present invention provides novel magnetic resonance imaging contrast agents which can detect physiological agents or target substances. The MRI agents of the invention are relatively inactive, or have weak relaxivity, as contrast enhancement agents in the absence of the physiological target substance, and are activated, thus altering the MR image, in the presence of the physiological target substance.

Viewed simplistically, this “trigger” mechanism, whereby the contrast agent is “turned on” (i.e. increases the relaxivity) by the presence of the target substance, is based on a dynamic equilibrium that affects the rate of exchange of water molecules in one or more coordination sites of a paramagnetic metal ion contained in the MRI contrast agents of the present invention. In turn, the rate of exchange of the water molecule is determined by the presence or absence of the target substance in the surrounding environment. Thus, in the absence of the target substance, the metal ion complexes of the invention which chelate the paramagnetic ion have reduced coordination sites available which can rapidly exchange with the water molecules of the local environment. In such a situation, the water coordination sites are substantially occupied or blocked by the coordination atoms of the chelator and at least one blocking moiety. Thus, the paramagnetic ion has essentially no water molecules in its “inner-coordination sphere”, i.e. actually bound to the metal when the target substance is absent. It is the interaction of the paramagnetic metal ion with the protons on the inner coordination sphere water molecules and the rapid exchange of such water molecules that cause the high observed relaxivity, and thus the imaging effect, of the paramagnetic metal ion. Accordingly, if all the coordination sites of the metal ion in the metal ion complex are occupied with moieties other than water molecules, as is the case when the target substance is absent, there is little if any net enhancement of the imaging signal by the metal ion complexes of the invention. However, when present, the target substance interacts with the blocking moiety or moities of the metal ion complex, effectively freeing at least one of the inner-sphere coordination sites on the metal ion complex. The water molecules of the local environment are then available to occupy the inner-sphere coordination site or sites, which will cause an increase in the rate of exchange of water and relaxivity of the metal ion complex toward water thereby producing image enhancement which is a measure of the presence of the target substance.

Generally, a 2 to 5% change in the MRI signal used to generate the image is sufficient to be detectable. Thus, it is preferred that the agents of the invention in the presence of a target substance increase the MRI signal by at least 2 to 5% as compared to the signal gain the absence of the target substance. Signal enhancement of 2 to 90% is preferred, and 10 to 50% is more preferred for each coordination site made available by the target substance interaction with the blocking moiety. That is, when the blocking moiety occupies two or more coordination sites, the release of the blocking moiety can result in double the increase in signal or more as compared to a single coordination site.

It should be understood that even in the absence of the target substance, at any particular coordination site, there will be a dynamic equilibrium for one or more coordination sites as between a coordination atom of the blocking moiety and water molecules. That is, even when a coordination atom is tightly bound to the metal, there will be some exchange of water molecules at the site. However, in most instances, this exchange of water molecules is neither rapid nor significant, and does not result in significant image enhancement. However, upon exposure to the target substance, the blocking moiety dislodges from the coordination site and the exchange of water is increased, i.e. rapid exchange and therefore an increase in relaxivity may occur, with significant image enhancement.

The complexes of the invention comprise a chelator and a blocking moiety. See for example U.S. Ser. Nos. 09/866,512; 09/972,302; 09/908,43609/715,859; and PCT/US01/14665, all of which are expressly incorporated by reference.

The metal ion complexes of the invention comprise a paramagnetic metal ion bound to a complex comprising a chelator and a blocking moiety. By “paramagnetic metal ion”, “paramagnetic ion” or “metal ion” herein is meant a metal ion which is magnetized parallel or antiparallel to a magnetic field to an extent proportional to the field. Generally, these are metal ions which have unpaired electrons; this is a term understood in the art. Examples of suitable paramagnetic metal ions, include, but are not limited to, gadolinium III (Gd+3 or Gd(III)), iron III (Fe+3 or Fe(III)), manganese II (Mn+2 or Mn(II)), yttrium III (Yt+3 or Yt(III)), dysprosium (Dy+3 or Dy(III)), and chromium (Cr(III) or Cr+3). In a preferred embodiment the paramagnetic ion is the lanthanide atom Gd(III), due to its high magnetic moment (u²=63 BM2), a symmetric electronic ground state (S8), and its current approval for diagnostic use in humans.

In addition to the metal ion, the metal ion complexes of the invention comprise a chelator and a blocking moiety which may be covalently attached to the chelator. Due to the relatively high toxicity of many of the paramagnetic ions, the ions are rendered nontoxic in physiological systems by binding to a suitable chelator. Thus, the substitution of blocking moieties in coordination sites of the chelator, which in the presence of the target substance are capable of vacating the coordination sites in favor of water molecules, may render the metal ion complex more toxic by decreasing the half-life of dissociation for the metal ion complex. Thus, in a preferred embodiment, only a single coordination site is occupied or blocked by a blocking moeity. However, for some applications, e.g. analysis of tissue and the like, the toxicity of the metal ion complexes may not be of paramount importance. Similarly, some metal ion complexes are so stable that even the replacement of one or more additional coordination atoms with a blocking moiety does not significantly effect the half-life of dissociation. For example, DOTA derivatives, described below, when complexed with Gd(III) is extremely stable. Accordingly, when a DOTA derivative serves as the chelator, several of the coordination atoms of the chelator may be replaced with blocking moieties without a significant increase in toxicity. Additionally such an agent would potentially produce a larger signal since it has two or more coordination sites which are rapidly exchanging water with the bulk solvent.

There are a variety of factors which influence the choice and stability of the chelate metal ion complex, including enthalpy and entropy effects (e.g. number, charge and basicity of coordinating groups, ligand field and conformational effects).

In general, the chelator has a number of coordination sites containing coordination atoms which bind the metal ion. The number of coordination sites, and thus the structure of the chelator, depends on the metal ion. The chelators used in the metal ion complexes of the present invention preferably have at least one less coordination atom (n−1) than the metal ion is capable of binding (n), since at least one coordination site of the metal ion complex is occupied or blocked by a blocking moeity, as described below, to confer functionality on the metal ion complex. Thus, for example, Gd(III) may have 8 strongly associated coordination atoms or ligands and is capable of weakly binding a ninth ligand. Accordingly, suitable chelators for Gd(III) will have less than 9 coordination atoms. In a preferred embodiment, a Gd(III) chelator will have 8 coordination atoms, with a blocking moiety either occupying or blocking the remaining site in the metal ion complex. In an alternative embodiment, the chelators used in the metal ion complexes of the invention have two less coordination atoms (n−2) than the metal ion is capable of binding (n), with these coordination sites occupied by one or more blocking moieties. Thus, alternative embodiments utilize Gd(ill) chelators with at least 5 coordination atoms, with at least 6 coordination atoms being preferred, at least 7 being particularly preferred, and at least 8 being especially preferred, with the blocking moiety either occupying or blocking the remaining sites. It should be appreciated that the exact structure of the chelator and blocking moiety may be difficult to determine, and thus the exact number of coordination atoms may be unclear. For example, it is possible that the chelator provide a fractional or non-integer number of coordination atoms; i.e. the chelator may provide 7.5 coordination atoms, i.e. the 8th coordination atom is on average not fully bound to the metal ion. However, the metal ion complex may still be functional, if the 8th coordination atom is sufficiently bound to prevent the rapid exchange of water at the site, and/or the blocking moiety impedes the rapid exchange of water at the site.

Accordingly, the present invention provides a number of suitable chelators for use in the present invention. The chelators shown below do not depict the chelated metal ion, although as will be appreciated by those in the art, they may be present as well.

In a preferred embodiment, the chelators have the structure depicted below:

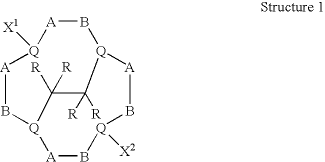

In this embodiment, each Q is independently selected from the group consisting of nitrogen, oxygen or sulfur. As will be appreciated by those in the art, the choice of these coordination atoms will depend in part on the metal chosen. When Gd+3 is used as the paramagnetic metal ion, nitrogen is preferred.

In addition, adjacent R groups on the “basket” structure can be joined to form aryl and cycloalkyl groups, including heteroaryl and heterocycloalkyl groups as generally outlined for other adjacent R groups discussed herein.

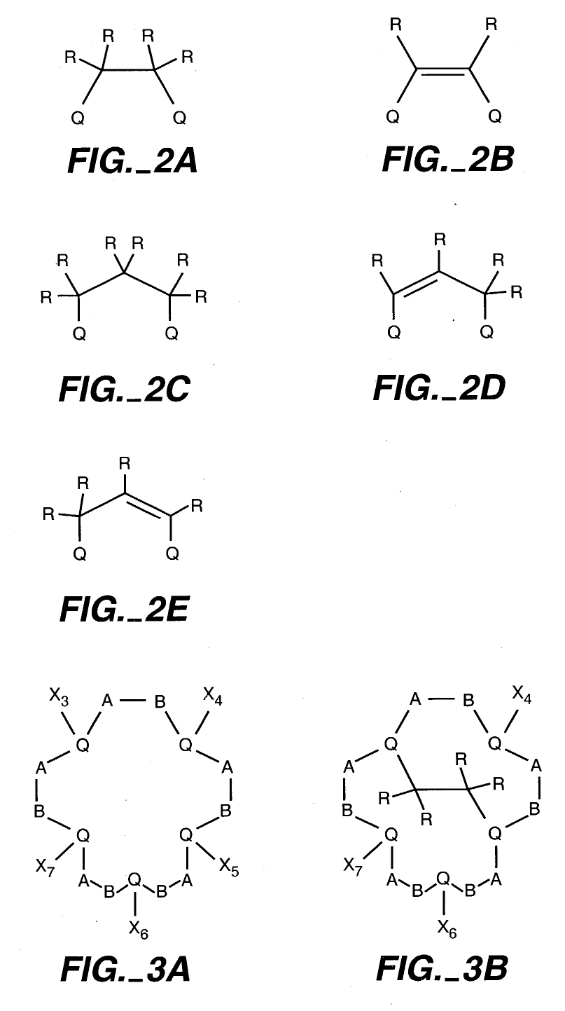

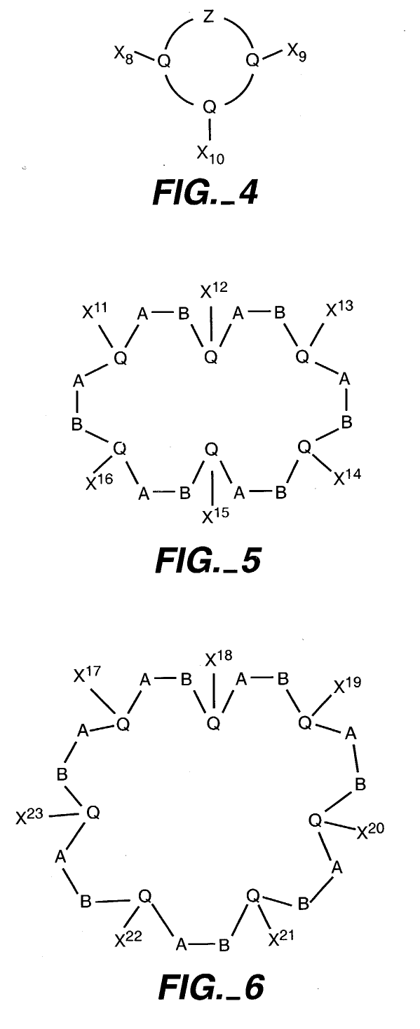

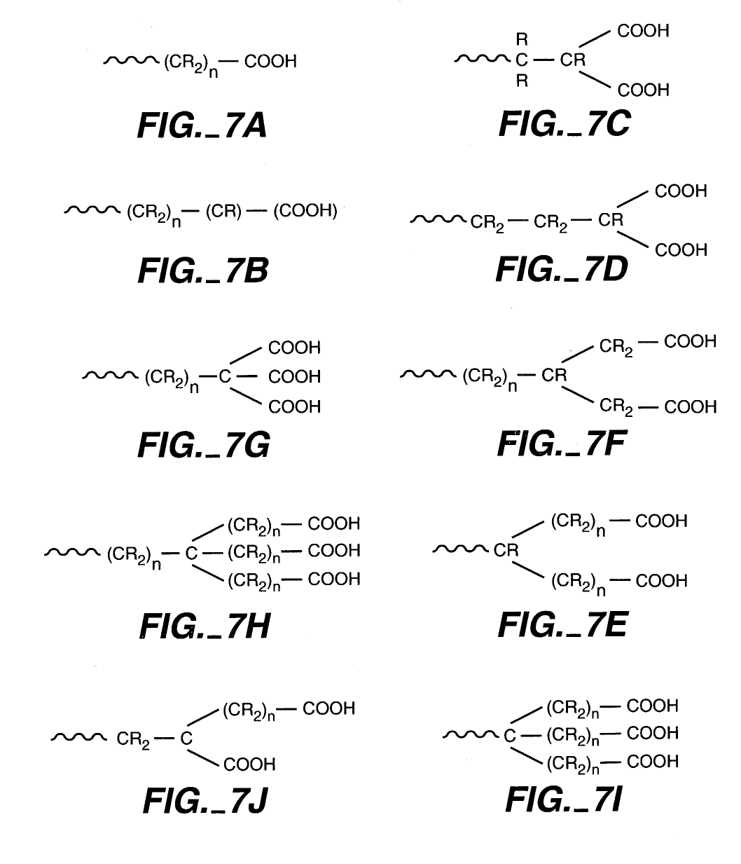

In a preferred embodiment, the —A—B— moiety is an alkyl moiety of C1-5, with alkyl and alkene bonds possible. In preferred embodiments, the —A—B— moiety is an alkyl of C2-3 selected from the group consisting of —CR₂—CR₂—, —CR═CR—, —CR₂—CR₂—CR₂—, —CR═CR—CR₂— and —CR₂—CR═CR— as is depicted in FIG. 2. The R substitution groups are as defined below.

The X groups of the invention (sometimes referred to herein as the “arms” of the chelator) generally provide one or more additional coordination atoms. Again, the choice of the coordination atom will depend on the metal ion used, with —COOH groups being a coordination moiety of preference with Gd+3. In some embodiments, straight alkyl chains (including both substituted alkyl, heteroalkyl and substituted heteroalkyl) are used. In alternate embodiments, particularly when X-linked embodiments as depicted herein (sometimes referred to as “basket” structures, due to the fact that on edge, the X-linking groups form a bicyclic-type or macro-bicyclic structure) the X groups depicted herein can be branched alkyl structures, with a single alkyl group comprising at least one carbon atom branching to provide multiple coordination atoms.

In addition, as more fully described below for Structures 2, 4 and 5, X groups may be attached together to form “basket” type structures.

In general, when not attached, the X groups of the Structures are independently selected from the group consisting of —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3. In this embodiment, n is an integer from 1 to 5, with 1, 2 and 3 being preferred; m is an integer from 0 to 5, with 0, 1 and 2 being preferred. BM is a blocking moiety as defined below.

It is to be understood that the exact composition of the X groups will depend on the presence of the metal ion. That is, the hydrogen atoms of the coordination group can be present in the absence of the metal ion, but are absent in the presence of the metal ion.

In a preferred embodiment at least one of the R groups attached to the “arms” of the chelator comprises an alkyl (including substituted and heteroalkyl groups), or aryl (including substituted and heteroaryl groups), i.e. is a group sterically bulkier than hydrogen. This is particular useful to drive the equilibrium towards “locking” the coordination atom of the arm into place to prevent water exchange, as is known for standard MRI contrast agents. Preferred groups include the C1 through C6 alkyl groups with methyl being particularly preferred.

This is particularly preferred when the blocking moiety is attached via one of the “arms”.

However the inclusion of too many groups may drive the equilibrium in the other direction effectively locking the coordination atom out of position. Therefore in a preferred embodiment only 1 or 2 of these positions is a non-hydrogen group, unless other methods are used to drive the equilibrium towards binding.

In the figures and agents described herein, R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, targeting moiety, blocking moiety, or, together with an adjacent R group forms an alkyl or aryl group.

As will be appreciated by those skilled in the art, many positions designated herein and in the structures of the figures may have more than 1 R group attached, depending on the valency of the atoms; generally, carbon atoms that are not participating in double bonds can have two R groups attached (R′ and R″), although in a preferred embodiment only a single non-hydrogen R group is attached at any particular position; that is, preferably at least one of the R groups at each position is hydrogen. Thus, if R is an alkyl or aryl group, there is generally an additional hydrogen attached to the carbon, although not depicted herein. In many preferred embodiments, all the R groups are hydrogen, with the exception of targeting and blocking moieties.

By “alkyl group” or grammatical equivalents herein is meant a straight or branched chain alkyl group, with straight chain alkyl groups being preferred. If branched, it may be branched at one or more positions, and unless specified, at any position. Also included within the definition of alkyl are heteroalkyl groups, wherein the heteroatom is selected from nitrogen, oxygen, phosphorus, sulfur and silicon. Also included within the definition of an alkyl group are cycloalkyl groups such as C5 and C6 rings, and heterocycloalkyl.

Additional suitable heterocyclic substituted rings are depicted in U.S. Pat. No. 5,087,440, expressly incorporated by reference. In some embodiments, two adjacent R groups may be bonded together to form ring structures together with the carbon atoms of the chelator, such as is described in U.S. Pat. No. 5,358,704, expressly incorporated by reference. These ring structures may be similarly substituted.

The alkyl group may range from about 1 to 20 carbon atoms (C1-C20), with a preferred embodiment utilizing from about 1 to about 10 carbon atoms (C1-C10), with about C1 through about C5 being preferred. However, in some embodiments, the alkyl group may be larger, for example when the alkyl group is the coordination site barrier.

By “alkyl amine” or grammatical equivalents herein is meant an alkyl group as defined above, substituted with an amine group at any position. In addition, the alkyl amine may have other substitution groups, as outlined above for alkyl group. The amine may be primary (—NH₂R), secondary (—NHR₂), or tertiary (—NR₃). When the amine is a secondary or tertiary amine, suitable R groups are alkyl groups as defined above. A preferred alkyl amine is p-aminobenzyl. When the alkyl amine serves as the coordination site barrier, as described below, preferred embodiments utilize the nitrogen atom of the amine as a coordination atom, for example when the alkyl amine includes a pyridine or pyrrole ring.

By “aryl group” or grammatical equivalents herein is meant aromatic aryl rings such as phenyl, heterocyclic aromatic rings such as pyridine, furan, thiophene, pyrrole, indole and purine, and heterocyclic rings with nitrogen, oxygen, sulfur or phosphorus.

Included within the definition of “alkyl” and “aryl” are substituted alkyl and aryl groups. That is, the alkyl and aryl groups may be substituted, with one or more substitution groups. For example, a phenyl group may be a substituted phenyl group. Suitable substitution groups include, but are not limited to, halogens such as chlorine, bromine and fluorine, amines, hydroxy groups, carboxylic acids, nitro groups, carbonyl and other alkyl and aryl groups as defined herein. Thus, arylalkyl and hydroxyalkyl groups are also suitable for use in the invention. Preferred substitution groups include alkyl amines and alkyl hydroxy.

In some embodiments, adjacent R groups can be joined to form cyclic structures, either cycloalkyl, cycloheteroalkyl, aryl, heteroaryl, or pluricyclic structures comprising a combination of these ring structures, including substituted derivatives of any of these. Similarly, when the R groups are alkyl and aryl groups, pluricyclic groups including cycloalkyl, cycloheteroalkyl, aryl, heteroaryl and substituted derivatives can also be used.

By “amino groups” or grammatical equivalents herein is meant —NH₂, —NHR and —NR₂ groups, with R being as defined herein.

By “nitro group” herein is meant an —NO₂ group.

By “sulfur containing moieties” herein is meant compounds containing sulfur atoms, including but not limited to, thia-, thio- and sulfo-compounds, thiols (—SH and —SR), and sulfides (—RSR—). By “phosphorus containing moieties” herein is meant compounds containing phosphorus, including, but not limited to, phosphines and phosphates. By “silicon containing moieties” herein is meant compounds containing silicon.

By “ether” herein is meant an —O—R group. Preferred ethers include alkoxy groups, with —O—(CH₂)₂CH₃ and —O—(CH₂)₄CH₃ being preferred.

By “ester” herein is meant a —COOR group.

By “halogen” herein is meant bromine, iodine, chlorine, or fluorine. Preferred substituted alkyls are partially or fully halogenated alkyls such as CF₃, etc.

By “aldehyde” herein is meant —RCHO groups.

By “alcohol” or “alkoxy” herein is meant —OH groups, and alkyl alcohols —ROH.

By “amido” herein is meant —RCONH— or RCONR— groups.

By “ethylene glycol” or “(poly)ethylene glycol” herein is meant a —(O—CH₂—CH₂)ₙ— group, although each carbon atom of the ethylene group may also be singly or doubly substituted, i.e. —(O—CR₂—CR₂)ₙ—, with R as described above. Ethylene glycol derivatives with other heteroatoms in place of oxygen (i.e. —(N—CH₂—CH₂)ₙ— or —(S—CH₂—CH₂)ₙ—, or with substitution groups) are also preferred.

By “ketone” herein is meant —R—CO—R—.

By “imino group” herein is meant —C—NH—C.

By “carbonyl” herein is meant —CO.

By “phosphorous moieties” herein is meant moieties containing the —PO(OH)(R)₂ group. The phosphorus may be an alkyl phosphorus; for example, DOTEP utilizes ethylphosphorus as a substitution group on DOTA. R is as defined above, with preferred embodiments utilizing alkyl, substituted alkyl and hydroxy. A preferred embodiment has a —PO(OH)₂R group.

In a preferred embodiment, the R group is a blocking moiety. In addition, at least one of the groups associated with the chelator (either an R group or a designated BM) is a blocking moiety. By “blocking moiety” or grammatical equivalents herein is meant a functional group associated with the chelator metal ion complexes of the invention which is capable of interacting with a target substance and which is capable, under certain circumstances, of substantially blocking the exchange of water in at least one inner coordination site of the metal ion of the metal ion complex. For example, when bound to or associated with the metal ion complexes of the invention, the blocking moiety occupies or blocks at least one coordination site of the metal ion in the absence of the target substance. Thus, the metal ion is coordinately saturated with the chelator and the blocking moiety or moieties in the absence of the target substance.

A blocking moiety may comprise several components. The blocking moiety has a functional moiety which is capable of interacting with a target substance, as outlined below. This functional moiety may or may not provide the coordination atom(s) of the blocking moiety. In addition, blocking moieties may comprise one or more linker groups to allow for correct spacing and attachment of the components of the blocking moiety. Furthermore, in the embodiment where the functional group of the blocking moiety does not contribute a coordination atom, the blocking moiety may comprise a coordination site barrier, which serves to either provide a coordination site atom or sterically prevent the rapid exchange of water at the coordination site; i.e. the coordination site barrier may either occupy or block the coordination site.

By “capable of interacting with a target substance” herein is meant that the blocking moiety has an affinity for the target substance, such that the blocking moiety will stop blocking or occupying at least one coordination site of the metal ion complex when the target substance is present. Thus, as outlined above, the blocking moiety is blocking or occupying at least one coordination site of the metal ion in the absence of the target substance. However, in the presence of the target substance, the blocking moiety associates or interacts with the target substance and is released from its association with the metal ion, thus freeing at least one coordination site of the metal ion such that the rapid exchange of water can occur at this site, resulting in image enhancement.

The nature of the interaction between the blocking moiety and the target substance will depend on the target substance to be detected or visualized via MRI. For example, suitable target substances include, but are not limited to, enzymes; proteins; peptides; nucleic acids; ions such as Ca+2, Mg+2, Zn+2, K+, Cl−, and Na+; cAMP; receptors such as cell-surface receptors and ligands; hormones; antigens; antibodies; ATP; NADH; NADPH; FADH₂; FNNH₂; coenzyme A (acyl CoA and acetyl CoA); and biotin, among others.

In some embodiments, the nature of the interaction is irreversible, such that the blocking moiety does not reassociate to block or occupy the coordination site; for example, when the blocking moiety comprises an enzyme substrate which is cleaved upon exposure to the target enzyme. Alternatively, the nature of the interaction is reversible, such that the blocking moiety will reassociate with the complex to hinder the exchange of water; for example, when the blocking moiety comprises an ion ligand, or a receptor ligand, as outlined below.

The corresponding blocking moieties will be enzyme substrates or inhibitors, receptor ligands, antibodies, antigens, ion binding compounds, substantially complementary nucleic acids, nucleic acid binding proteins, etc.

In a preferred embodiment, the target substance is an enzyme, and the blocking moiety is an enzyme substrate. In this embodiment, the blocking moiety is cleaved from the metal ion complex of the invention, allowing the exchange of water in at least one coordination site of the metal ion complex. This embodiment allows the amplification of the image enhancement since a single molecule of the target substance is able to generate many activated metal ion complexes, i.e. metal ion complexes in which the blocking moiety is no longer occupying or blocking a coordination site of the metal ion.

As will be appreciated by those skilled in the art, the possible enzyme target substances are quite broad. The target substance enzyme may be chosen on the basis of a correlation to a disease condition, for example, for diagnositic purposes. Alternatively, the metal ion complexes of the present invention may be used to establish such correlations.

Suitable classes of enzymes include, but are not limited to, hydrolases such as proteases, carbohydrases, lipases and nucleases; isomerases such as racemases, epimerases, tautomerases, or mutases; transferases, kinases and phophatases.

As will be appreciated by those skilled in the art, the potential list of suitable enzyme targets is quite large. Enzymes associated with the generation or maintenance of arterioschlerotic plaques and lesions within the circulatory system, inflammation, wounds, immune response, tumors, may all be detected using the present invention. Enzymes such as lactase, maltase, sucrase or invertase, cellulase, α-amylase, aldolases, glycogen phosphorylase, kinases such as hexokinase, proteases such as serine, cysteine, aspartyl and metalloproteases may also be detected, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain;; metalloproteinases including MMP-1 through MMP-10, particularly MMP-1, MMP-2, MMP-7 and MMP-9; and caspases, such as caspase-3, -5, -8 and other caspases of the apoptotic pathway, and interleukin-converting enzyme (ICE). Similarly, bacterial and viral infections may be detected via characteristic bacterial and viral enzymes. As will be appreciated in the art, this list is not meant to be limiting.

Once the target enzyme is identified or chosen, enzyme substrate blocking moieties can be designed using well known parameters of enzyme substrate specificities.

For example, when the enzyme target substance is a protease, the blocking moieity may be a peptide or polypeptide which is capable of being cleaved by the target protease. By “peptide” or “polypeptide” herein is meant a compound of about 2 to about 15 amino acid residues covalently linked by peptide bonds. Preferred embodiments utilize polypeptides from about 2 to about 8 amino acids, with about 2 to about 4 being the most preferred. Preferably, the amino acids are naturally occurring amino acids, although amino acid analogs and peptidomimitic structures are also useful. Under certain circumstances, the peptide may be only a single amino acid residue.

Preferred target substance/peptide blocking moiety pairs include, but are not limited to, cat B and GGGF; cat B and GFQGVQFAGF; cat B and GFGSVGFAGF; cat B and GLVGGAGAGF; cat B and GGFLGLGAGF; cat D and GFGSTFFAGF; caspase-3 and DEVD; MMP-7 and PELR; MMP-7 and PLGLAR; MMP-7 and PGLWA-(D-arg); MMP-7 and PMALWMR; and MMP-7 and PMGLRA.

Similarly, when the enzyme target substance is a carbohydrase, the blocking moiety will be a carbohydrate group which is capable of being cleaved by the target carbohydrase. For example, when the enzyme target is lactase or β-galactosidase, the enzyme substrate blocking moiety is lactose or galactose. Similar enzyme/blocking moiety pairs include sucrase/sucrose, maltase/maltose, and α-amylase/amylose. In addition, the addition of carbohydrate moieties such as galactose, outlined herein, can alter the biodistribution of the agents; for example, the galactose blocking moieties outlined herein cause concentration in liver, kidneys and spleen.

In another embodiment, the blocking moiety may be an enzyme inhibitor, such that in the presence of the enzyme, the inhibitor blocking moiety disassociates from the metal ion complex to interact or bind to the enzyme, thus freeing an inner coordination sphere site of the metal ion for interaction with water. As above, the enzyme inhibitors are chosen on the basis of the enzyme target substance and the corresponding known characteristics of the enzyme.

In a preferred embodiment, the blocking moiety is a phosphorus moiety, as defined above, such as —(OPO(OR₂))ₙ, wherein n is an integer from 1 to about 10, with from 1 to 5 being preferred and 1 to 3 being particularly preferred. Each R is independently hydrogen or a substitution group as defined herein, with hydrogen being preferred. This embodiment is particularly useful when the target molecule is alkaline phosphatase or a phosphodiesterase, or other enzymes known to cleave phosphorus containing moieties such as these.

In one embodiment, the blocking moiety is a nucleic acid. The nucleic acid may be single-stranded or double stranded, and includes nucleic acid analogs such as peptide nucleic acids and other well-known modifications of the ribose-phosphate backbone, such as phosphorthioates, phosphoramidates, morpholino structures, etc. The target molecule can be a substantially complementary nucleic acid or a nulceic acid binding moiety, such as a protein.

In a preferred embodiment, the target substance is a physiological agent. As for the enzyme/substrate embodiment, the physiological agent interacts with the blocking moiety of the metal ion complex, such that in the presence of the physiological agent, there is rapid exchange of water in at least one inner sphere coordination site of the metal ion complex. Thus, the target substance may be a physiologically active ion, and the blocking moiety is an ion binding ligand. For example, as shown in the Examples, the target substance may be the Ca+2 ion, and the blocking moiety may be a calcium binding ligand such as is known in the art (see Grynkiewicz et al., J. Biol. Chem. 260(6):3440-3450 (1985); Haugland, R. P., Molecular Probes Handbook of Fluorescent Probes and Research Chemicals (1989-1991)). Other suitable target ions include Mn+2, Mg+2, Zn+2, Na+, and Cl−.

When Ca+2 is the target substance, preferred blocking moieties include, but are not limited to, the acetic acid groups of bis(o-amino-phenoxy)ethane-N,N,N′,N′-tetraacetic acid (BAPTA), ethylene glycol bis(β-aminoethyl ether)-N,N,N′,N′-tetraacetic acid (EGTA); ethylenediaminetetracetic acid (EDTA); and derivatives thereof, such as disclosed in Tsien, Biochem. 19:2396-2404 (1980). Other known chelators of Ca+2 and other divalent ions, such as quin2 (2-[[2-[bis(carboxymethyl) amino]-5-methyphenoxy]methyl-6-methoxy-8-[bis(carboxymethyl)amino]quinoline; fura-1, fura-2, fura-3, stil-1, stil-2 and indo-1 (see Grynkiewicz et al., supra).

As for the enzyme/substrate embodiments, the metabolite may be associated with a particular disease or condition within an animal. For example, as outlined below, BAPTA-DOTA derivatives may be used to diagnose Alzeheimer's disease and other neurological disorders.

In a preferred embodiment, the blocking moiety is a ligand for a cell-surface receptor or is a ligand which has affinity for a extracellular component. In this embodiment, as for the physiological agent embodiment, the ligand has sufficient affinity for the metal ion to prevent the rapid exchange of water molecules in the absence of the target substance. Alternatively, there may be R groups “locking” the ligand into place, as described herein, resulting in either the contribution of a coordination atom or that the ligand serves as a coordination site barrier. In this embodiment the ligand blocking moiety has a higher affinity for the target substance than for the metal ion. Accordingly, in the presence of the target substance, the ligand blocking moiety will interact with the target substance, thus freeing up at least one coordination site in the metal ion complex and allowing the rapid exchange of water and an increase in relaxivity. Additionally, in this embodiment, this may result in the accumulation of the MRI agent at the location of the target, for example at the cell surface. This may be similar to the situation where the blocking moiety is an enzyme inhibitor, as well.

In a preferred embodiment, the blocking moiety is a photocleavable moiety. That is, upon exposure to a certain wavelength of light, the blocking moiety is cleaved, allowing an increase in the exchange rate of water in at least one coordination site of the complex. This embodiment has particular use in developmental biology fields (cell lineage, neuronal development, etc.), where the ability to follow the fates of particular cells is desirable. Suitable photocleavable moieties are similar to “caged” reagents which are cleaved upon exposure to light. A particularly preferred class of photocleavable moieties are the O-nitrobenzylic compounds, which can be synthetically incorporated into a blocking moiety via an ether, thioether, ester (including phosphate esters), amine or similar linkage to a heteroatom (particularly oxygen, nitrogen or sulfur). Also of use are benzoin-based photocleavable moieties. A wide variety of suitable photocleavable moieties is outlined in the Molecular Probes Catalog, supra.

The blocking moiety itself may block or occupy at least one coordination site of the metal ion. That is, one or more atoms of the blocking moiety (i.e. the enzyme substrate, ligand, moiety which interacts with a physiological agent, photocleavable moiety, etc.) itself serves as a coordination atom, or otherwise blocks access to the metal ion by steric hinderance. For example, it appears that one or more of the atoms of the galactose blocking moiety outlined in the Examples may be direct coordination atoms for the Gd(III) metal ion. Similarly, peptide based blocking moieties for protease targets may contribute coordination atoms.

In an alternative embodiment, the blocking moiety further comprises a “coordination site barrier” which is covalently tethered to the complex in such a manner as to allow disassociation upon interaction with a target substance. For example, it may be tethered by one or more enzyme substrate blocking moieties. In this embodiment, the coordination site barrier blocks or occupies at least one of the coordination sites of the metal ion in the absence of the target enzyme substance. Coordination site barriers are used when coordination atoms are not provided by the functional portion of the blocking moiety, i.e. the component of the blocking moiety which interacts with the target substance. The blocking moiety or moieties such as an enzyme substrate serves as the tether, covalently linking the coordination site barrier to the metal ion complex. In the presence of the enzyme target, the enzyme cleaves one or more of the enzyme substrates, either within the substrate or at the point of attachment to the metal ion complex, thus freeing the coordination site barrier. The coordination site or sites are no longer blocked and the bulk water is free to rapidly exchange at the coordination site of the metal ion, thus enhancing the image. As will be appreciated by those in the art, a similar result can be accomplished with other types of blocking moieties.

In one embodiment, the coordination site barrier is attached to the metal ion complex at one end. When the enzyme target cleaves the substrate blocking moiety, the coordination site barrier is released. In another embodiment, the coordination site barrier is attached to the metal ion complex with more than one substrate blocking moiety. The enzyme target may cleave only one side, thus removing the coordination site barrier and allowing the exchange of water at the coordination site, but leaving the coordination site barrier attached to the metal ion complex. Alternatively, the enzyme may cleave the coordination site barrier completely from the metal ion complex.

In a preferred embodiment, the coordination site barrier occupies at least one of the coordination sites of the metal ion. That is, the coordination site barrier contains at least one atom which serves as at least one coordination atom for the metal ion. In this embodiment, the coordination site barrier may be a heteroalkyl group, such as an alkyl amine group, as defined above, including alkyl pyridine, alkyl pyrroline, alkyl pyrrolidine, and alkyl pyrole, or a carboxylic or carbonyl group. The portion of the coordination site barrier which does not contribute the coordination atom may also be consider a linker group.

In an alternative embodiment, the coordination site barrier does not directly occupy a coordination site, but instead blocks the site sterically. In this embodiment, the coordination site barrier may be an alkyl or substituted group, as defined above, or other groups such as peptides, proteins, nucleic acids, etc.

In this embodiment, the coordination site barrier is preferrably linked via two enzyme substrates to opposite sides of the metal ion complex, effectively “stretching” the coordination site barrier over the coordination site or sites of the metal ion complex.

In some embodiments, the coordination site barrier may be “stretched” via an enzyme substrate on one side, covalently attached to the metal ion complex, and a linker moeity, as defined below, on the other. In an alternative embodiment, the coordination site barrier is linked via a single enzyme substrate on one side; that is, the affinity of the coordination site barrier for the metal ion is higher than that of water, and thus the blocking moiety, comprising the coordination site barrier and the enzyme substrate, will block or occupy the available coordination sites in the absence of the target enzyme.

In some embodiments, the metal ion complexes of the invention have a single associated or bound blocking moiety. In such embodiments, the single blocking moiety impedes the exchange of water molecules in at least one coordination site. Alternatively, as is outlined below, a single blocking moiety may hinder the exchange of water molecules in more than one coordination site, or coordination sites on different chelators.

In alternative embodiments, two or more blocking moieties are associated with a single metal ion complex, to implede the exchange of water in at least one or more coordination sites.

It should be appreciated that the blocking moieties of the present invention may further comprise a linker group as well as a functional blocking moiety. That is, blocking moieties may comprise functional blocking moieties in combination with a linker group and/or a coordination site barrier.

Linker groups will be used to optimize the steric considerations of the metal ion complex. That is, in order to optimize the interaction of the blocking moiety with the metal ion, linkers may be introduced to allow the functional blocking moiety to block or occupy the coordination site. In general, the linker group is chosen to allow a degree of structural flexibility. For example, when a blocking moiety interacts with a physiological agent which does not result in the blocking moiety being cleaved from the complex, the linker must allow some movement of the blocking moiety away from the complex, such that the exchange of water at at least one coordination site is increased.

Generally, suitable linker groups include, but are not limited to, alkyl and aryl groups, including substituted alkyl and aryl groups and heteroalkyl (particularly oxo groups) and heteroaryl groups, including alkyl amine groups, as defined above. Preferred linker groups include p-aminobenzyl, substituted p-aminobenzyl, diphenyl and substituted diphenyl, alkyl furan such as benzylfuran, carboxy, and straight chain alkyl groups of 1 to 10 carbons in length. Particularly preferred linkers include p-aminobenzyl, methyl, ethyl, propyl, butyl, pentyl, hexyl, acetic acid, propionic acid, aminobutyl, p-alkyl phenols, 4-alkylimidazole. The selection of the linker group is generally done using well known molecular modeling techniques, to optimize the obstruction of the coordination site or sites of the metal ion.

In a preferred embodiment, a coordination site barrier can be attached by a cleavable linker as outlined herein. A preferred embodiment utilizes esterase linkages. Esterase linkages are particularly preferred when the blocking moiety is attached via an “arm” of the chelate, as the product of an esterase reaction is a carboxylic acid, which thus allows the regeneration of a stable chelate. Alternatively, cleavable peptide linkers can also be used.

The blocking moiety is attached to the metal ion complex in a variety of ways. In a preferred embodiment, as noted above, the blocking moiety is attached to the metal ion complex via a linker group. Alternatively, the blocking moiety is attached directly to the metal ion complex; for example, as outlined below, the blocking moiety may be a substituent group on the chelator.

The blocking moieties are chosen and designed using a variety of parameters. In the embodiment which uses a coordination site barrier, i.e. when the functional group of the blocking moiety does not provide a coordination atom, and the coordination site barrier is fastened or secured on two sides, the affinity of the coordination site barrier of the blocking moiety for the metal ion complex need not be great, since it is tethered in place. That is, in this embodiment, the complex is “off” in the absence of the target substance. However, in the embodiment where the blocking moiety is linked to the complex in such a manner as to allow some rotation or flexibility of the blocking moiety, for example, it is linked on one side only, the blocking moiety should be designed such that it occupies the coordination site a majority of the time.

When the blocking moiety is not covalently tethered on two sides, it should be understood that blocking moieties and coordination site barriers are chosen to maximize three basic interactions that allow the blocking moiety to be sufficiently associated with the complex to hinder the rapid exchange of water in at least one coordination site of the complex. First, there may be electrostatic interactions between the blocking moiety and the metal ion, to allow the blocking moiety to associate with the complex. Secondly, there may be Van der Waals and dipole-dipole interactions. Thirdly, there may be ligand interactions, that is, one or more functionalities of the blocking moiety may serve as coordination atoms for the metal. In addition, linker groups may be chosen to force or favor certain conformations, to drive the equilibrium towards an associated blocking moiety. Similarly, removing degrees of fredom in the molecule may force a particular conformation to prevail.

Furthermore, effective “tethering” of the blocking moiety down over the metal ion may also be done by engineering in other non-covalent interactions that will serve to increase the affinity of the blocking moiety to the chelator complex, as is depicted below.

Potential blocking moieties may be easily tested to see if they are functional; that is, if they sufficiently occupy or block the appropriate coordination site or sites of the complex to prevent rapid exchange of water. Thus, for example, complexes are made with potential blocking moieties and then compared with the chelator without the blocking moiety in imaging experiments. Once it is shown that the blocking moiety is a sufficient “blocker”, the target substance is added and the experiments repeated, to show that interaction with the target substance increases the exchange of water and thus enhances the image.

In addition, the complexes and metal ion complexes of the invention may further comprise one or more targeting moieties; i.e. one or more R groups may be a targeting moiety. That is, a targeting moiety may be attached at any of the R positions (or to a linker, including a polymer, or to a blocking moiety, etc.), although in a preferred embodiment the targeting moiety does not replace a coordination atom. By “targeting moiety” herein is meant a functional group which serves to target or direct the complex to a particular location, cell type, diseased tissue, or association. In general, the targeting moiety is directed against a target molecule. As will be appreciated by those in the art, the MRI contrast agents of the invention are generally injected intraveneously; thus preferred targeting moieties are those that allow concentration of the agents in a particular localization. Thus, for example, antibodies, cell surface receptor ligands and hormones, lipids, sugars and dextrans, alcohols, bile acids, fatty acids, amino acids, peptides and nucleic acids may all be attached to localize or target the contrast agent to a particular site.

In a preferred embodiment, the targeting moiety allows targeting of the MRI agents of the invention to a particular tissue or the surface of a cell. That is, in a preferred embodiment the MRI agents of the invention need not be taken up into the cytoplasm of a cell to be activated.

As will be appreciated by those in the art, the targeting moieties can be attached in a large number of different ways, and in a variety of configurations.

In a preferred embodiment, the targeting moiety is a peptide. For example, chemotactic peptides have been used to image tissue injury and inflammation, particularly by bacterial infection; see WO 97/14443, hereby expressly incorporated by reference in its entirety.

In a preferred embodiment, the targeting moiety is an antibody. The term “antibody” includes antibody fragments, as are known in the art, including Fab Fab₂, single chain antibodies (Fv for example), chimeric antibodies, etc., either produced by the modification of whole antibodies or those synthesized de novo using recombinant DNA technologies.

In a preferred embodiment, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986); Riechmann et al., Nature 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321:522-525 (1986); Riechmann et al., Nature 332:323-327 (1988); Verhoeyen et al., Science 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol. 227:381 (1991); Marks et al., J. Mol. Biol. 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology, 14:826 (1996); Lonberg and Huszar, Intern. Rev. Immunol. 13:65-93 (1995).

Bispecific antibodies are monoclonal, preferably human or humanized, antibodies that have binding specificities for at least two different antigens. In the present case, one of the binding specificities is for a first target molecule and the other one is for a second target molecule.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology 121:210 (1986).

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In a preferred embodiment, the antibody is directed against a cell-surface marker on a cancer cell; that is, the target molecule is a cell surface molecule. As is known in the art, there are a wide variety of antibodies known to be differentially expressed on tumor cells, including, but not limited to, HER2, VEGF, etc.

In addition, antibodies against physiologically relevant carbohydrates may be used, including, but not limited to, antibodies against markers for breast cancer (CA15-3, CA 549, CA 27.29), mucin-like carcinoma associated antigen (MCA), ovarian cancer (CA125), pancreatic cancer (DE-PAN-2), and colorectal and pancreatic cancer (CA 19, CA 50, CA242).

In a preferred embodiment, the targeting moiety is all or a portion (e.g. a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF-α and TGF-β), EPO receptor (EPO), TPO receptor (TPO), ciliary neurotrophic factor receptor, prolactin receptor, and T-cell receptors. In particular, hormone ligands are preferred. Hormones include both steroid hormones and proteinaceous hormones, including, but not limited to, epinephrine, thyroxine, oxytocin, insulin, thyroid-stimulating hormone, calcitonin, chorionic gonadotropin, cortictropin, follicle-stimulating hormone, glucagon, leuteinizing hormone, lipotropin, melanocyte-stimutating hormone, norepinephrine, parathryroid hormone, thyroid-stimulating hormone (TSH), vasopressin, en kephal ins, seratonin, estradiol, progesterone, testosterone, cortisone, and glucocorticoids and the hormones listed above. Receptor ligands include ligands that bind to receptors such as cell surface receptors, which include hormones, lipids, proteins, glycoproteins, signal transducers, growth factors, cytokines, and others.

In a preferred embodiment, the targeting moiety is a carbohydrate. By “carbohydrate” herein is meant a compound with the general formula Cx(H2O)y. Monosaccharides, disaccharides, and oligo- or polysaccharides are all included within the definition and comprise polymers of various sugar molecules linked via glycosidic linkages. Particularly preferred carbohydrates are those that comprise all or part of the carbohydrate component of glycosylated proteins, including monomers and oligomers of galactose, mannose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates. In particular, polysaccharides (including, but not limited to, arabinogalactan, gum arabic, mannan, etc.) have been used to deliver MRI agents into cells; see U.S. Pat. No. 5,554,386, hereby incorporated by reference in its entirety.

In a preferred embodiment, the targeting moiety is a lipid. “Lipid” as used herein includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

In addition, as will be appreciated by those in the art, any moiety which may be utilized as a blocking moiety can be used as a targeting moiety. Particularly preferred in this regard are enzyme inhibitors, as they will not be cleaved off and will serve to localize the MRI agent in the location of the enzyme.

In a preferred embodiment, the targeting moiety may be used to either allow the internalization of the MRI agent to the cell cytoplasm or localize it to a particular cellular compartment, such as the nucleus.

In a preferred embodiment, the targeting moiety is all or a portion of the HIV-1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); and Baldin et al., EMBO J. 9:1511 (1990), all of which are incorporated by reference.

In a preferred embodiment, the targeting moiety is a nuclear localization signal (NLS). NLSs are generally short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-β nuclear localization signal (ARRRRP); NFκB p50 (EEVQRKRQKL; Ghosh et al., Cell 62:1019 (1990); NFκB p65 (EEKRKRTYE; Nolan et al., Cell 64:961 (1991); and others (see for example Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gin Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

In a preferred embodiment, targeting moieties for the hepatobiliary system are used; see U.S. Pat. Nos. 5,573,752 and 5,582,814, both of which are hereby incorporated by reference in their entirety

In a preferred embodiment, the chelates of the invention have the formula depicted in Structure 2:

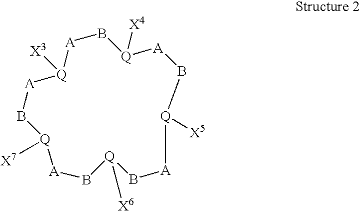

As noted above, in this embodiment, any two (or in some cases three) X groups can also be joined together to form “basket” type structures. In this embodiment, the X groups generally form alkyl or aryl groups, including heteroalkyl, substituted alkyl, heteroaryl and substituted aryl groups. In the case of alkyl groups, straight chain alkyl groups of C1-5 are preferred.

In this embodiment, two metal ions may be added to the chelate. In this case, the X groups are designed to provide a sufficient number of coordination atoms, and preferably (although not required) one blocking moiety is used, such that the interaction of the blocking moiety with the target substance causes water exchange in the inner coordination sites of the two metal ions.

In a preferred embodiment, the chelates of the invention have the formula depicted in Structure 3:

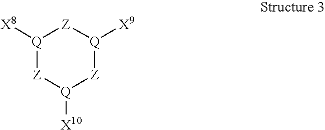

In this embodiment, each Z is —(CR₂)n-wherein n is at least 1 and R is a substitution group; at least two of X¹—X³ are selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3.

In Structure 3, at least one X group or an R substitution group comprises a blocking moiety.

In a preferred embodiment, the chelates of the invention have the formula depicted in Structure 4:

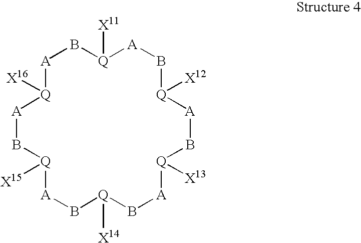

In Structure 4, each X₁—X₇ are independently selected from the group consisting of hydrogen, R, blocking moiety,—(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3. Optionally, two or more of X1—X7 are joined to form a C1-5 alkyl group. Furthermore, in this embodiment, eitherX11, X12, X13, X14, X15 or X16 comprises a BM, or at least one R comprises a BM.

In a preferred embodiment, the chelates of the invention have the formula depicted in Structure 5:

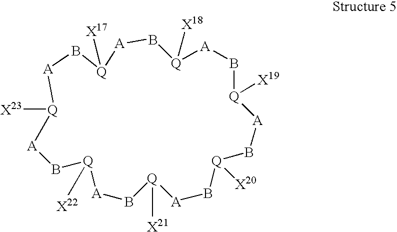

In Structure 5, each of X₁₇—X₂₃ are independently selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3. Optionally two ore more of of X17—X23 are joined to form a C1-10 alkyl group, and either X17, X18, X19, X20, X21, X22 or X23 comprises a BM, or at least one R comprises a BM.

In a preferred embodiment, the metal ion complexes of the present invention are water soluble or soluble in aqueous solution. By “soluble in aqueous solution” herein is meant that the MRI agent has appreciable solubility in aqueous solution and other physiological buffers and solutions. Solubility may be measured in a variety of ways. In one embodiment, solubility is measured using the United States Pharmacopeia solubility classifications, with the metal ion complex being either very soluble (requiring less than one part of solvent for 1 part of solute), freely soluble (requiring one to ten parts solvent per 1 part solute), soluble (requiring ten to thirty parts solvent per 1 part solute), sparingly soluble (requiring 30 to 100 parts solvent per 1 part solute), or slightly soluble (requiring 100-1000 parts solvent per 1 part solute).

Testing whether a particular metal ion complex is soluble in aqueous solution is routine, as will be appreciated by those in the art. For example, the parts of solvent required to solubilize a single part of MRI agent may be measured, or solubility in gm/ml may be determined.

In a preferred embodiment, the MRI contrast agents of the invention comprise more than one metal ion, such that the signal is increased. As is outlined below, this may be done in a number of ways, including, but not limited to, the use of multiple metal ions in a single chelate, the use of a single blocking moiety to block more than one chelated metal ion, or the oligomerization of the agents of the invention, including both multimers and the use of polymeric linkers to attach agents together.

In a preferred embodiment, particularly in the case of the “larger” chelates of the invention, a single chelate may have more than one metal ion associated. This can be done using the correct spacing and the correct number of coordination atoms and/or blocking moieties supplied on the “arms” or on the macrocycle, particularly in order to maximize the stability of the complex in cases for administration to patients.

In a preferred embodiment, the MRI agents of the invention comprise at least two paramagnetic metal ions, each with a chelator and blocking moiety; that is, multimeric MRI agents are made. In a preferred embodiment, the chelators are linked together, either directly or through the use of a linker such as a coupling moiety or polymer. For example, using substitution groups that serve as functional groups for chemical attachment on the chelator, attachment to other chelators may be accomplished. As will be appreciated by those in the art, attachment of more than one MRI agent may also be done via the blocking moieties (or coordination site barriers, etc.), although these are generally not preferred.

In a preferred embodiment, the chelators of the invention include one or more substitution groups that serve as functional groups for chemical attachment. Suitable functional groups include, but are not limited to, amines (preferably primary amines), carboxy groups, and thiols (including SPDP, alkyl and aryl halides, maleimides, α-haloacetyls, and pyridyl disulfides) are useful as functional groups that can allow attachment.

In one embodiment, the chelators are linked together directly, using at least one functional group on each chelator. This may be accomplished using any number of stable bifunctional groups well known in the art, including homobifunctional and heterobifunctional linkers (see Pierce Catalog and Handbook, 1994, pages T155-T200, hereby expressly incorporated by reference). This may result in direct linkage, for example when one chelator comprises a primary amine as a functional group and the second comprises a carboxy group as the functional group, and carbodiimide is used as an agent to activate the carboxy for attach by the nucleophilic amine (see Torchilin et al., Critical Rev. Therapeutic Drug Carrier Systems, 7(4):275-308 (1991). Alternatively, as will be appreciated by those in the art, the use of some bifunctional linkers results in a short coupling moiety being present in the structure. A “coupling moiety” is capable of covalently linking two or more entities. In this embodiment, one end or part of the coupling moiety is attached to the first MRI contrast agent, and the other is attached to the second MRI agent. The functional group(s) of the coupling moiety are generally attached to additional atoms, such as alkyl or aryl groups (including hetero alkyl and aryl, and substituted derivatives), to form the coupling moiety. Oxo linkers are also preferred. As will be appreciated by those in the art, a wide range of coupling moieties are possible, and are generally only limited by the ability to synthesize the molecule and the reactivity of the functional group. Generally, the coupling moiety comprises at least one carbon atom, due to synthetic requirements; however, in some embodiments, the coupling moiety may comprise just the functional group.

In a preferred embodiment, the coupling moiety comprises additional atoms as a spacer. As will be appreciated by those in the art, a wide variety of groups may be used. For example, a coupling moiety may comprise an alkyl or aryl group substituted with one or more functional groups. Thus, in one embodiment, a coupling moiety containing a multiplicity of functional groups for attachment of multiple MRI contrast agents may be used, similar to the polymer embodiment described below. For example, branched alkyl groups containing multiple functional groups may be desirable in some embodiments.

In an additional embodiment, the linker is a polymer. In this embodiment, a polymer comprising at least one MRI contrast agent of the invention is used. As will be appreciated by those in the art, these MRI contrast agents may be monomeric (i.e. one metal ion, one chelator, one blocking moiety) or a duplex, as is generally described below (i.e. two metal ions, two chelators, one blocking moiety). Preferred embodiments utilize a plurality of MRI agents per polymer. The number of MRI agents per polymer will depend on the density of MRI agents per unit length and the length of the polymer.

The character of the polymer will vary, but what is important is that the polymer either contain or can be modified to contain functional groups for the the attachment of the MRI contrast agents of the invention. Suitable polymers include, but are not limited to, functionalized dextrans, styrene polymers, polyethylene and derivatives, polyanions including, but not limited to, polymers of heparin, polygalacturonic acid, mucin, nucleic acids and their analogs including those with modified ribose-phosphate backbones, the polypeptides polyglutamate and polyaspartate, as well as carboxylic acid, phosphoric acid, and sulfonic acid derivatives of synthetic polymers; and polycations, including but not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, spermine, spermidine and polypeptides such as protamine, the histone polypeptides, polylysine, polyarginine and polyornithine; and mixtures and derivatives of these. Particularly preferred polycations are polylysine and spermidine, with the former being especially preferred. Both optical isomers of polylysine can be used. The D isomer has the advantage of having long-term resistance to cellular proteases. The L isomer has the advantage of being more rapidly cleared from the subject. As will be appreciated by those in the art, linear and branched polymers may be used.

A preferred polymer is polylysine, as the —NH₂ groups of the lysine side chains at high pH serve as strong nucleophiles for multiple attachment of activated chelating agents. At high pH the lysine monomers are coupled to the MRI agents under conditions that yield on average 5-20% monomer substitution.

In some embodiments, particularly when charged polymers are used, there may be a second polymer of opposite charge to the first that is electrostatically associated with the first polymer, to reduce the overall charge of polymer-MRI agent complex. This second polymer may or may not contain MRI agents.

The size of the polymer may vary substantially. For example, it is known that some nucleic acid vectors can deliver genes up to 100 kilobases in length, and artificial chromosomes (megabases) have been delivered to yeast. Therefore, there is no general size limit to the polymer. However, a preferred size for the polymer is from about 10 to about 50,000 monomer units, with from about 2000 to about 5000 being particularly preferred, and from about 3 to about 25 being especially preferred.

It should be understood that the multimeric MRI agents of the invention may be made in a variety of ways, including those listed above. What is important is that manner of attachment does not significantly alter the functionality of the agents; that is, the agents must still be “off” in the absence of the target substance and “on” in its presence.

In a preferred embodiment, the MRI contrast agents of the invention are “duplexes”. In this embodiment, the MRI duplex comprises two chelators, each with a paramagnetic metal ion, and at least one blocking moiety that restricts the exchange of water in at least one coordination site of each chelator. In this way, a sort of signal amplification occurs, with two metal ions increasing the signal with a single target molecule. While “duplex” implies two chelators, it is intended to refer to complexes comprising a single blocking moiety donating coordination atoms to more than 1 metal ion/chelator complex. As will be appreciated by those in the art, the MRI agents of this embodiment may have a number of different conformations.

As outlined above, the MRI duplex moieties may also be combined into higher multimers, either by direct linkage or via attachment to a polymer.

In a preferred embodiment, the chelator and the blocking moiety are covalently linked; that is, the blocking moiety is a substitution group on the chelator. In this embodiment, the substituted chelator, with the bound metal ion, comprises the metal ion complex which in the absence of the target substance has all possible coordination sites occupied or blocked; i.e. it is coordinatively saturated.

In an alternative embodiment, the chelator and the blocking moiety are not covalently attached. In this embodiment, the blocking moiety has sufficient affinity for the metal ion to prevent the rapid exchange of water molecules in the absence of the target substance. However, in this embodiment the blocking moiety has a higher affinity for the target substance than for the metal ion. Accordingly, in the presence of the target substance, the blocking moiety will have a tendency to be dislodged from the metal ion to interact with the target substance, thus freeing up a coordination site in the metal ion complex and allowing the rapid exchange of water and an increase in relaxivity.

What is important is that the metal ion complex, comprising the metal ion, the chelator and the blocking moiety, is not readily able to rapidly exchange water molecules when the blocking moeities are in the inner coordination sphere of the metal ion, such that in the absence of the target substance, there is less or little substantial image enhancement.

The complexes of the invention are generally synthesized using well known techniques. See, for example, Moi et al., supra; Tsien et al., supra; Borch et al., J. Am. Chem. Soc., p2987 (1971); Alexander, (1995), supra; Jackels (1990), supra, U.S. Pat. Nos. 5,155,215, 5,087,440, 5,219,553, 5,188,816, 4,885,363, 5,358,704, 5,262,532; Meyer et al., (1990), supra, Moi et al., (1988), and McMurray et al., Bioconjugate Chem. 3(2):108-117 (1992)). Cross-bridged tetraazamacrocycles have been shown to form complexes with transition metal ions having unprecedented kinetic stability, even though they do not fully saturate the metal's coordination sites. See Hubin et al., J. Chem. Soc. Chem, Commun, 1998:1675; Hubin et al., Inorg. Chem, 1999, 38:4435; Hubin et al., J. Am. Chem. Soc. 2000, 122:2512; WO 98/39098 and WO 98/39046, all of which are expressly incorporated by reference.

The contrast agents of the invention are complexed with the appropriate metal ion as is known in the art. While the structures depicted herein all comprise a metal ion, it is to be understood that the contrast agents of the invention need not have a metal ion present initially. Metal ions can be added to water in the form of an oxide, a halide or acetate and treated with an equimolar amount of a contrast agent composition. The contrast agent may be added as an aqueous solution or suspension. Dilute acid or base can be added if need to maintain a neutral pH. Heating at temperatures as high as 100° C. may be required.

The complexes of the invention can be isolated and purified, for example using HPLC systems.

Pharmaceutical compositions comprising pharmaceutically acceptable salts of the contrast agents can also be prepared by using a base to neutralize the complexes while they are still in solution. Some of the complexes are formally uncharged and do not need counterions.

Once synthesized, the metal ion complexes of the invention have use as magnetic resonance imaging contrast or enhancement agents. Specifically, the functional MRI agents of the invention have several important uses. First, they may be used to diagnose disease states of the brain, as is outlined below. Second, they may be used in real-time detection and differentiation of myocardial infraction versus ischemia. Third, they may be used in in vivo, i.e. whole organism, investigation of antigens and immunocytochemistry for the location of tumors. Fourth, they may be used in the identification and localization of toxin and drug binding sites. In addition, they may be used to perform rapid screens of the physiological response to drug therapy.

The metal ion complexes of the invention may be used in a similar manner to the known gadolinium MRI agents. See for example, Meyer et al., supra; U.S. Pat. Nos. 5,155,215; 5,087,440; Margerstadt et al., Magn. Reson. Med. 3:808 (1986); Runge et al., Radiology 166:835 (1988); and Bousquet et al., Radiology 166:693 (1988). The metal ion complexes are administered to a cell, tissue or patient as is known in the art. A “patient” for the purposes of the present invention includes both humans and other animals and organisms, such as experimental animals. Thus the methods are applicable to both human therapy and veterinary applications. In addition, the metal ion complexes of the invention may be used to image tissues or cells; for example, see Aguayo et al., Nature 322:190 (1986).

Generally, sterile aqueous solutions of the contrast agent complexes of the invention are administered to a patient in a variety of ways, including orally, intrathecally and especially intraveneously in concentrations of 0.003 to 1.0 molar, with dosages from 0.03, 0.05, 0.1, 0.2, and 0.3 millimoles per kilogram of body weight being preferred. Dosages may depend on the structures to be imaged. Suitable dosage levels for similar complexes are outlined in U.S. Pat. Nos. 4,885,363 and 5,358,704.

In addition, the contrast agents of the invention may be delivered via specialized delivery systems, for example, within liposomes (see Navon, Magn. Reson. Med. 3:876-880 (1986)) or microspheres, which may be selectively taken up by different organs (see U.S. Pat. No. 5,155,215).

In some embodiments, it may be desirable to increase the blood clearance times (or half-life) of the MRI agents of the invention. This has been done, for example, by adding carbohydrate polymers to the chelator (see U.S. Pat. No. 5,155,215). Thus, one embodiment utilizes polysaccharides as substitution R groups on the compositions of the invention.

A preferred embodiment utilizes complexes which cross the blood-brain barrier. Thus, as is known in the art, a DOTA derivative which has one of the carboxylic acids replaced by an alcohol to form a neutral DOTA derivative has been shown to cross the blood-brain barrier. Thus, for example, neutral complexes are designed that cross the blood-brain barrier with blocking moieties which detect Ca+2 ions. These compounds are used in MRI of a variety of neurological disorders, including Alzeheimer's disease. Currently it is difficult to correctly diagnosis Alzeheimer's disease, and it would be useful to be able to have a physiological basis to distinguish Alzeheimer's disease from depression, or other treatable clinical symptoms for example.

All references cited herein are incorporated by reference.

We claim:

1. A chelate having the formula: 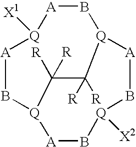
wherein
each Q is independently selected from the group consisting of nitrogen, oxygen or sulfur;
A—B is a structure selected from the group consisting of —CR₂—CR₂—, —CR═CR—, —CR₂—CR₂—CR₂—, —CR═CR—CR₂— and —CR₂—CR═CR—;
X1 and X2 are independently selected from the group consisting of CR₂COO⁻, CR₂COOH, CR₂(BM), CR(CR₂COO⁻)₂, CR(CR₂COO⁻)(BM), CR(CR₂COOH)₂ and CR(CR₂COOH)(BM), wherein BM is a blocking moiety; and
each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, targeting moiety, blocking moiety, or, together with an adjacent R group forms an alkyl or aryl group;
wherein either:
a) X¹ or X² comprises a BM; or
b) at least one R comprises a BM.

2. A chelate according to claim 1 wherein said BM is a peptide.

3. A chelate according to claim 1 wherein said BM is a carbohydrate.

4. A chelate according to claim 1 wherein each Q is a nitrogen.

5. An MRI composition comprising the chelate of claim 1 complexed with a paramagnetic ion.

6. An MRI composition according to claim 5 wherein said paramagnetic ion is Gd+3.

7. A chelate having the formula: 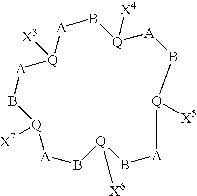
wherein
each Q is independently selected from the group consisting of nitrogen, oxygen or sulfur;
A—B is a structure selected from the group consisting of —CR₂—CR₂—, —CR═CR—, —CR₂—CR₂—CR₂—, —CR═CR—CR₂— and —CR₂—CR═CR—;
X³, X⁴, X5, X6 and X⁷ are independently selected from the group consisting of —(CR₂)nCOO⁻, —(CR₂₎ₙCOOH, (CR₂)n(BM), —CR(CR₂COO⁻)₂, —CR(CR₂COO⁻) (BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3, wherein BM is a blocking moiety;
each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, blocking moiety, or, together with an adjacent R group forms an alkyl or aryl group;
wherein optionally two of X³—X⁷ are joined to form —CR₂—CR₂—, —CR═CR—, —CR₂—CR₂—CR₂— or —CR═CR—CR₂—; and
wherein either:
a) X³, X⁴, X⁵, X⁶ or X⁷ comprises a BM; or
b) at least one R comprises a BM.

8. A chelate according to claim 7 wherein said BM is a peptide.

9. A chelate according to claim 7 wherein said BM is a carbohydrate.

10. A chelate according to claim 7 wherein each Q is a nitrogen.

11. An MRI composition comprising the chelate of claim 7 complexed with a paramagnetic ion.

12. An MRI composition according to claim 11 wherein said paramagnetic ion is Gd+3.

13. A chelate having the formula: 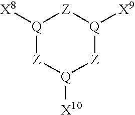
wherein
each Q is independently selected from the group consisting of nitrogen, sulfur or oxygen;
each Z is —(CR₂)n- wherein n is at least 1 and R is a substitution group;
at least two of X⁸—X¹⁰ are selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3;
wherein at least one R or X comprises a blocking moiety.

14. A chelate according to claim 13 wherein said BM is a peptide.

15. A chelate according to claim 13 wherein said BM is a carbohydrate.

16. A chelate according to claim 13 wherein each Q is a nitrogen.

17. An MRI composition comprising the chelate of claim 13 complexed with a paramagnetic ion.

18. An MRI composition according to claim 17 wherein said paramagnetic ion is Gd+3.

19. A chelate having the formula: 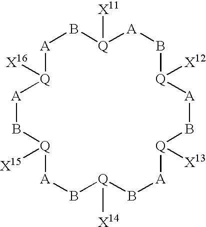
wherein
A and B are selected from the group consisting of CR₂—CR₂, CR═CR, CR₂—CR₂—CR₂, CR═CR—CR₂, and CR₂—CR═CR;
each Q is independently selected from the group consisting of nitrogen, sulfur or oxygen;
each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, blocking moiety, or, together with an adjacent R group forms an alkyl or aryl group, or together with an non adjacent R group forms an alkyl or aryl group;
wherein X¹¹—X¹⁶ are selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3;
wherein optionally two of X¹—X⁷ are joined to form a C1-5 alkyl group;
wherein either:
a) X¹¹, X¹², X¹³, X¹⁴, X¹⁵ or X¹⁶ comprises a BM; or
b) at least one R comprises a BM.

20. A chelate according to claim 19 wherein said BM is a peptide.

21. A chelate according to claim 19 wherein said BM is a carbohydrate.

22. A chelate according to claim 19 wherein each Q is a nitrogen.

23. An MRI composition comprising the chelate of claim 19 complexed with a paramagnetic ion.

24. An MRI composition according to claim 19 wherein said paramagnetic ion is Gd+3.

25. A chelate having the formula: 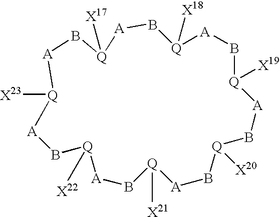
wherein
A and B are selected from the group consisting of CR₂—CR₂, CR═CR, CR₂—CR₂—CR₂, CR═CR—CR₂, and CR₂—CR═CR;
each Q is independently selected from the group consisting of nitrogen, sulfur or oxygen;
each R is independently selected from the group consisting of hydrogen, alkyl, aryl, alcohol, amine, amido, nitro, ether, ester, ketone, imino, aldehyde, alkoxy, carbonyl, halogen, sulfur containing moiety, phosphorus containing moiety, blocking moiety, or, together with an adjacent R group forms an alkyl or aryl group, or together with an non adjacent R group forms an alkyl or aryl group;
wherein each of X²³ are independently selected from the group consisting of hydrogen, R, blocking moiety, —(CR₂)nCOO⁻, —(CR₂₎ₙCOOH, (CR₂)n(BM), —CR(CR₂COO⁻)₂, —CR(CR₂COO⁻)(BM), —CR(CR₂COOH)₂ and —CR(CR₂COOH)(BM), —(CR₂)n-CR((CR₂)m-COOH)2, —(CR₂)n-CR((CR₂)m-COO⁻)2, —(CR₂)m-CR[((CR₂)m-COOH))((CR₂)m-BM), —(CR₂)n-C(CR₂)m-COOH)3; and —C((CR₂)n-COOH)3;
wherein optionally two of X¹⁷—X²³ are joined to form a C1-10 alkyl group;
wherein either:
a) X¹⁷, X¹⁸, X¹⁹, X²⁰, X²¹, X²² or X²³ comprises a BM; or
b) at least one R comprises a BM.

26. A chelate according to claim 25 wherein said BM is a peptide.

27. A chelate according to claim 25 wherein said BM is a carbohydrate.

28. A chelate according to claim 25 wherein each Q is a nitrogen.

29. An MRI composition comprising the chelate of claim 25 complexed with a paramagnetic ion.

30. An MRI composition according to claim 29 wherein said paramagnetic ion is Gd+3.

31. A method of imaging a cell, tissue or patient comprising administering the composition according to claim 1, 7, 19 or 25 and acquiring an MRI image.


SEQUENCE LISTING (14 sequences)

SEQ ID NO: 1 - protein, 10 aa, Artificial Sequence
  synthetic.
GFQGVQFAGF

SEQ ID NO: 2 - protein, 10 aa, Artificial Sequence
  synthetic
GFGSVGFAGF

SEQ ID NO: 3 - protein, 10 aa, Artificial Sequence
  synthetic
GLVGGAGAGF

SEQ ID NO: 4 - protein, 10 aa, Artificial Sequence
  synthetic
GGFLGLFAGF

SEQ ID NO: 5 - protein, 10 aa, Artificial Sequence
  synthetic
GFGSTFFAGF

SEQ ID NO: 6 - protein, 6 aa, Artificial Sequence
  synthetic
PLGLAR

SEQ ID NO: 7 - protein, 5 aa, Artificial Sequence
  synthetic
PGLWA

SEQ ID NO: 8 - protein, 7 aa, Artificial Sequence
  synthetic
PMALWMR

SEQ ID NO: 9 - protein, 6 aa, Artificial Sequence
  synthetic
PMGLRA

SEQ ID NO: 10 - protein, 7 aa, Simian virus 40
PKKKRKV

SEQ ID NO: 11 - protein, 6 aa, Homo sapiens
ARRRRP

SEQ ID NO: 12 - protein, 10 aa, Mus musculus
EEVQRKRQKL

SEQ ID NO: 13 - protein, 9 aa, Mus musculus
EEKRKRTYE

SEQ ID NO: 14 - protein, 20 aa, Xenopus laevis
AVKRPAATKKAGQAKKKKLD